(19) United States
(12) Patent Application Publication
(10) Pub. No.: US 2007/0154409 A1
(43) Pub. Date: Jul. 5, 2007

(54) Chewable tooth paste tablet

(76) Inventor: David Annis, Toronto (CA)

Correspondence Address: Elias C. Borges, Suite 406, 555 Burnhamthorpe Road, Toronto, ON, M9C 2Y3, CA

(21) Appl. No.: 11/325,544

(22) Filed: Jan. 5, 2006

(51) Int. Cl. A61K 8/19

(52) U.S. Cl. 424/49

(57) ABSTRACT

The present invention is a method of brushing teeth consisting of the steps of placing a tooth cleansing tablet into the user's month, chewing the tablet into a paste and then manually brushing the teeth with the paste using a tooth brush. The tooth cleansing tablet consists of a quantity of tooth powder formed into a firm tablet. The tablet is dimensioned and configured to be easily inserted into the mouth and broken up into a paste by chewing.

### FIELD OF THE INVENTION

The invention relates generally to pastes and powders for brushing teeth.

### BACKGROUND OF THE INVENTION

Tooth cleaning agents to assist users in brushing their teeth are generally available in a paste form. These tooth pastes generally consist of a mild tooth abrasive such as baking soda or chalk in combination with water, stabilizers, thickening agents, coloring agents and flavorings. In a great majority of cases, these tooth pastes are packaged in squeezable tubes made of plastic or metal. To dispense the tooth paste, the user simply squeezes the tube. Alternatively, the pastes can be packaged in small bottles provided with a pump for pumping out the paste as desired. Prior to the widespread use of tooth paste, tooth cleaning powers were readily used. These tooth powders generally consisted of a dry mixture of mild tooth abrasives with flavor enhancing ingredients. Tooth powders were commonly packaged in small tins. Both tooth paste and tooth powders required the user to apply a quantity of the tooth paste or tooth powder to the tooth brush before using the tooth brush to clean the teeth.

Over the years, numerous devices and methods have been used to store and dispense tooth paste. Regardless of the storage and dispensing device used, the user first had to apply the tooth paste to the brush before using the product. Also, by far the highest cost associated with producing a tube or bottle of tooth paste is the cost of packaging. An improved form of tooth cleaning product which avoids the costs of packaging would be a cost effective alternative to tooth paste.

### SUMMARY OF THE INVENTION

In accordance with the present invention, there is provided a method of brushing teeth consisting of the steps of placing a tooth cleansing tablet into the user's month, chewing the tablet into a paste and then manually brushing the teeth with the paste using a tooth brush. The tooth cleansing tablet consists of a quantity of tooth powder formed into a firm tablet. The tablet is dimensioned and configured to be easily inserted into the mouth and broken up into a paste by chewing.

With the foregoing in view, and other advantages as will become apparent to those skilled in the art to which this invention relates as this specification proceeds, the invention is herein described by reference to the accompanying drawings forming a part hereof, which includes a description of the preferred typical embodiment of the principles of the present invention.

### DESCRIPTION OF THE DRAWINGS

FIG. 1. is a cross-sectional view of a tooth paste tablet made in accordance with the invention.

In the drawings like characters of reference indicate corresponding parts in the different figures.

### DETAILED DESCRIPTION OF THE INVENTION

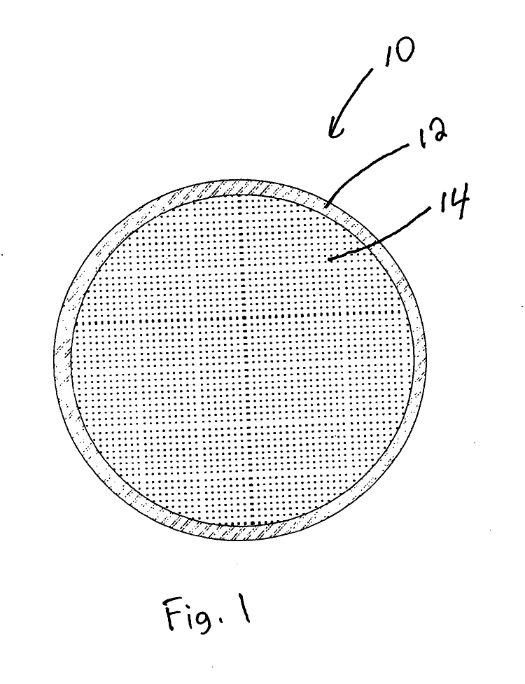

Referring firstly to FIG. 1, a tooth cleaning tablet used in the present invention and shown generally as item 10, consists of a tablet of tooth powder 14 surrounded by a coating 12. Tablet 14 is made from a combination of a mild tooth abrasive with a binding agent and flavoring. The tooth abrasive and the binding agent are selected such that the mixture can be formed into pellets or tablets simply by pressing the tooth powder in a standard pelletizing machine. It will be appreciated that there are numerous tooth abrasives which can be used in making the tooth powder. Indeed, virtually any substantially inert food grade powdered material which is granular and gritty can be used as the tooth abrasive. Without limiting the generality of the sort of powders which may be used as a tooth abrasive for this invention, commonly used tooth cleaning powders used in manufacturing common tooth paste can be used, including baking soda powder, chalk and the like. A binding agent is preferably added to the tooth abrasive to assist the mixture in forming firm pellets. Numerous commercially available food grade binders can be used, including glycerin, water, alginates, gelatin and the like. For the finished tablet to have a pleasant flavor, flavoring agents such as peppermint extract, vanilla extract, corn syrup, or the like are added.

The tooth powder tablet 14 is preferably formed by pressing a quantity of tooth powder in a mold to form a firm pellet or tablet. Pelletizing machines (not shown) capable of producing large numbers of pellets or tablets are readily available in the market. The composition of the tooth powder and the pressure used to form the tooth powder into pellets are selected to ensure that the resulting tablets are firm enough to be packaged together and physically handled, yet not so firm as to prevent the tablet from being comfortably chewed into a paste.

Tooth powder tablet 14 is protected by coating 12 which acts to protect the integrity of the tooth powder tablet and also to provide a measure of water resistance. Coating 12 can be made of any food grade ingredient commonly used to form the coatings on candies or gum balls. Without limiting the generality of the possible ingredients used in forming the coating, commonly available ingredients often used to form coatings include corn zein, food grade shellac, carnauba wax, whey proteins, gelatin and even starch. Preferably, coloring agents can be added to the coating to make the finished product more appealing. The coating is applied to a thickness of less than about 1 mm, although a thicker coating may be applied if a more “crunchy” finished tablet is desired. The coating can be done in a tumbler by periodically applying the liquified coating material to a large quantity of tablets while the tablets are gently tumbled.

The size of the finished tablet is preferably between 10 mm in diameter to 15 mm in diameter, which represents a volume of tooth powder roughly equivalent to that required for brushing one's teeth. The finished product can be packaged in quantities of a few tablets to hundreds of tablets in simple plastic bags. The finished tablets can also be dispensed in gum ball machines or the like.

The method of the present invention will now be discussed with reference to FIG. 2. User 20 first places a tooth cleaning tablet 10 into his/her mouth 22. The user then chews on the tablet until the tablet is broken into small pieces. The broken pieces of the tablet forms a paste in the user's mouth as the tablet is chewed. When the paste reaches the desired consistency, the user then simply brushes his/her teeth with the paste using a standard tooth brush 24.

The present invention has several advantages over the prior art. Firstly, the product packaging costs are quite minimal resulting in a very cost effective product. Additionally, the product can be formed as brightly colored spheres or tablets which resemble candy. The tablets can also be made into a variety of pleasing shapes such as cubes or even small figures such as animals or cartoon characters. These bright colors and pleasing shapes make the cleaning tablets more palatable to small children, making it easier for parents to encourage their children to brush their teeth.

A specific embodiment of the present invention has been disclosed; however, several variations of the disclosed embodiment could be envisioned as within the scope of this invention. It is to be understood that the present invention is not limited to the embodiments described above, but encompasses any and all embodiments within the scope of the following claims.

Therefore, what is claimed is:

1. A tooth cleansing agent for use in combination with a tooth brush comprising a quantity of a tooth powder formed into a firm tablet, the tablet being dimensioned and configured to be easily broken up into a paste by chewing.

2. The tooth cleansing agent described in claim 1 wherein the tablet is coated with a coating made of a food grade material.

3. The tooth cleansing agent described in claim 2 wherein the food grade material making up the coating is selected from the group consisting of corn zein, food-grade shellac, wax, gelatin and protein.

4. The tooth cleansing agent described in claim 1 wherein the tooth powder is made from a mixture of a binding agent plus a tooth abrasive and cleanser, the tooth abrasive and cleanser selected from the group consisting of baking soda, chalk, and salt.

5. The tooth cleansing agent described in claim 1 wherein the tooth powder is made from a mixture of a tooth abrasive plus a binding agent, the binding agent and tooth abrasive being selected to permit the tooth powder to be formed into the firm tablet by pressing.

6. The tooth cleansing agent described in claim 5 wherein the tablet is coated with a coating made of a food grade material.

7. The tooth cleansing agent described in claim 6 wherein the food grade material forming the coating is selected from the group consisting of corn zein, food-grade shellac, wax, gelatin and protein.

8. The tooth cleansing agent described in claim 5 wherein the tooth abrasive and cleanser is selected from the group consisting of baking soda, chalk and salt.

9. The tooth cleansing agent described in claim 8 wherein the tablet is between about 10 mm to about 15 mm in diameter and wherein the coating has a thickness of less than 1 mm.

10. A method of brushing teeth in a user's mouth comprising the steps of:
a) placing a tooth cleansing tablet into the user's mouth, the tablet comprising a quantity of a tooth powder formed into a firm tablet, the tablet being dimensioned and configured to be easily inserted into the mouth and broken up into a paste by chewing the tablet;
b) chewing the tablet into a paste, and
c) manually brushing the teeth with the paste with the aid of a tooth brush.

11. The method of claim 10 wherein the tablet is coated with a coating made of a food grade material.

12. The method of claim 11 wherein the food grade material making up the coating is selected from the group consisting of corn zein, food-grade shellac, wax, gelatin and protein.

13. The method of claim 10 wherein the tooth powder is made from a mixture of a binding agent plus a tooth abrasive and cleanser, the tooth abrasive and cleanser selected from the group consisting of baking soda, chalk, and salt.

14. The method of claim 10 wherein the tooth powder is made from a mixture of a tooth abrasive plus a binding agent, the binding agent and tooth abrasive being selected to permit the tooth powder to be formed into the firm tablet by pressing.

15. The method of claim 14 wherein the tablet is coated with a coating made of a food grade material.

16. The method of claim 14 wherein the food grade material forming the coating is selected from the group consisting of corn zein, food-grade shellac, wax, gelatin and protein.

17. The method of claim 14 wherein the tooth abrasive and cleanser is selected from the group consisting of baking soda, chalk and salt.

18. The method of claim 17 wherein the tablet is between about 10 mm to about 15 mm in diameter and wherein the coating has a thickness of less than 1 mm.

19. A method of brushing teeth in a user's mouth comprising the steps of:
a) placing a tooth cleansing tablet into the user's month, the tablet comprising a quantity of a tooth powder coated by a coating made of a food grade material, the tooth powder comprising a mixture of a tooth abrasive with a binding agent, the tooth abrasive and binding agent selected to permit the mixture to be formed into the tablet by pressing, the tablet being dimensioned and configured to be easily inserted into the mouth and broken up into a paste by chewing the tablet;
b) chewing the tablet into a paste, and
c) manually brushing the teeth with the paste with the aid of a tooth brush.

20. The method of claim 19 wherein the tooth abrasive and cleanser is selected from the group consisting of baking soda, chalk and salt and wherein the food grade material forming the coating is selected from the group consisting of corn zein, food-grade shellac, wax, gelatin and protein.
